SEQUENCE LISTING (22 sequences)

SEQ ID NO: 1 - DNA, 23 bp, Artificial Sequence
  Primer for PCR amplification of CYP1A1 gene
cctccaggatccctacactgatc

SEQ ID NO: 2 - DNA, 22 bp, Artificial Sequence
  Primer for the PCR amplification of CYP1A1 gene
cccggatcccagatagcaaaac

SEQ ID NO: 3 - DNA, 27 bp, Artificial Sequence
  Primer for PCR amplification of CYP1A2 gene
gcaggtaccgttggtaaagatggcatt

SEQ ID NO: 4 - DNA, 27 bp, Artificial Sequence
  Primer for PCR amplification of CYP1A2 gene
agccatggaccggagtcttaccaccac

SEQ ID NO: 5 - DNA, 26 bp, Artificial Sequence
  Primer for PCR amplification of CYP2A6 gene
cccgaattcaccatgctggcctcagg

SEQ ID NO: 6 - DNA, 26 bp, Artificial Sequence
  Primer for PCR amplification of CYP2A6 gene
ccgaattccagacctgcaccggcaca

SEQ ID NO: 7 - DNA, 27 bp, Artificial Sequence
  Primer for PCR amplification of CYP2B6 gene
cagggatcccagaccaggaccatggaa

SEQ ID NO: 8 - DNA, 28 bp, Artificial Sequence
  Primer for PCR amplification of CYP2B6 gene
tttgggatccttccctcagccccttcag

SEQ ID NO: 9 - DNA, 28 bp, Artificial Sequence
  Primer for PCR amplification of CYP2C8 gene
ggggtaccttcaatggaaccttttgtgg

SEQ ID NO: 10 - DNA, 26 bp, Artificial Sequence
  Primer for PCR amplification of CYP2C8 gene
cccaagcttgcattcttcagacaggg

SEQ ID NO: 11 - DNA, 30 bp, Artificial Sequence
  Primer for PCR amplification of CYP2C9 gene
ggaattcggcttcaatggattctcttgtgg

SEQ ID NO: 12 - DNA, 28 bp, Artificial Sequence
  Primer for PCR amplification of CYP2C9 gene
cgtctagacttcttcagacaggaatgaa

SEQ ID NO: 13 - DNA, 26 bp, Artificial Sequence
  Primer for PCR amplification of CYP2C18 gene
cccgaattcaccatgctggcctcagg

SEQ ID NO: 14 - DNA, 26 bp, Artificial Sequence
  Primer for PCR amplification of CYP2C18 gene
ccgaattccagacctgcaccggcaca

SEQ ID NO: 15 - DNA, 21 bp, Artificial Sequence
  Primer for PCR amplification of CYP2C19 gene
atggatccttttgtggtcctt

SEQ ID NO: 16 - DNA, 19 bp, Artificial Sequence
  Primer for PCR amplification of CYP2C19 gene
agcagccagaccatctgtg

SEQ ID NO: 17 - DNA, 22 bp, Artificial Sequence
  Primer for PCR amplification of CYP2D6 gene
ctaagggaacgacactcatcac

SEQ ID NO: 18 - DNA, 22 bp, Artificial Sequence
  Primer for PCR amplification of CYP2D6 gene
ctcaccaggaaagcaaagacac

SEQ ID NO: 19 - DNA, 26 bp, Artificial Sequence
  Primer for PCR amplification of CYP3A5 gene
gttgaagaatccaagtggcgatggac

SEQ ID NO: 20 - DNA, 27 bp, Artificial Sequence
  Primer for PCR amplification of CYP3A5 gene
acagaatccttgaagaccaaagtagaa

SEQ ID NO: 21 - DNA, 27 bp, Artificial Sequence
  Primer for PCR amplification of GST(A1) gene
ccaggatcctgctatcatggcagagaa

SEQ ID NO: 22 - DNA, 33 bp, Artificial Sequence
  Primer for PCR amplification of GST(A1) gene
tatggatcccaaaactttagaacattggtattg

(19) United States
(12) Patent Application Publication
(10) Pub. No.: US 2005/0176147 A1
(43) Pub. Date: Aug. 11, 2005

(54) Method for obtaining a cell model capable of reproducing in vitro the metabolic idiosyncrasy of humans

(75) Inventors: Jose Vicente Castell Ripoll, Barcelona (ES); Ramiro Jover Atienza, Barcelona (ES); Maria Jose Gomez Lechon, Barcelona (ES)

Correspondence Address: MICHAEL BEST & FRIEDRICH LLC, 401 NORTH MICHIGAN AVENUE, SUITE 1900, CHICAGO, IL, 60611-4212, US

(73) Assignee: ADVANCED IN VITRO CELL TECHNOLOGIES, S.L., Barcelona (ES)

(21) Appl. No.: 10/775,914

(22) Filed: Feb. 10, 2004

(51) Int. Cl. C12Q 1/68; C12N 5/08; C12N 15/88

(52) U.S. Cl. 435/458; 435/366

(57) ABSTRACT

A cell model is disclosed that has a phenotypic profile, expressing at least one drug biotransformation enzyme. This model includes a cell having cytochrome reductase activity, transformed with at least one expression vector comprising a DNA sequence for a drug biotransformation enzyme. The method is based on the use of expression vectors coding for the sense and anti-sense mRNA of the Phase I and Phase II drug biotransformation enzymes showing a greatest variability in humans for transforming cells expressing cytochrome reductase activity. Such vectors can modulate (increase or decrease) the individualised expression of an enzyme without affecting the other enzymes. This cell model can reproduce in vitro the metabolic idiosyncrasy of humans. It is applicable in the study of development of new drugs, specifically in the study of metabolism, potential idiosyncratic hepatotoxicity, medicament interactions, etc., of new drugs.

### FIELD OF THE INVENTION

A cell model is disclosed that has a phenotypic profile, expressing at least one drug biotransformation enzyme. This model includes a cell having cytochrome reductase activity, transformed with at least one expression vector comprising a DNA sequence for a drug biotransformation enzyme.

### BACKGROUND OF THE INVENTION

It is known that drug metabolism is the leading cause of the variability of clinical responses in humans. Drugs, in addition to exerting a pharmacological action on a given target tissue, undergo chemical transformations during their transit through the organism (absorption, distribution and excretion). This process is known as drug metabolism or biotransformation, and can take place in all organs or tissues with which the drug is in contact. The process is catalysed by a group of enzymes generically known as drug metabolisation or biotransformation enzymes, mainly present in the microsomal and/or cytosolic cell fractions, and to a lesser extent in the extracellular space, which include various oxygenases, oxidases, hydrolases and conjugation enzymes (Garattini 1994). In this context, the liver is the most relevant organ, and monooxygenases dependent on the P450 (CYP450) cytochrome together with flavin-monooxygenases, cytochrome C reductase, UDP-glucuronyl transferase and glutathione transferase are the enzymes most directly involved (Watkins 1990). The intestine, lungs, skin and kidney follow in importance as regards their ability to metabolise xenobiotics including drugs (Krishna 1994). These biotransformation processes can also be performed by the saprophytic microorganisms colonising the intestinal tract:

The phenomenon of biotransformation is crucial in the context of drug bioavailability, variability of pharmacological response and toxicity, and understanding it is vital for an improved medicament use and development. In fact, biotransformation is the most variable stage and that which affects most the plasma drug levels after administration to various individuals. The rate at which a drug is biotransformed and the number and abundance of the various metabolites formed (metabolic profile) can vary greatly among individuals, which is an explanation for the observation that for some a given drug dose can be therapeutically effective, as it generates adequate plasma levels, while for others it is ineffective as a faster metabolisation does not allow obtaining the therapeutic plasma concentration. The situation is even more serious in individuals lacking one of the enzymes involved in the drug metabolism, who attain plasma levels much higher than the expected levels after a dose that is tolerated well by the rest of the population (Meyer 1997).

The great variability in drug and xenobiotic metabolism among human population groups/individuals has been confirmed numerous times (Shimada et al 1994). Two factors are mainly responsible for these differences: the inducibility of biotransformation enzymes by xenobiotics and the existence of gene polymorphisms.

Indeed, one of the characteristics of biotransformation enzymes is that they can be induced by xenobiotics, so that exposure to these compounds results in a greater expression of the enzymes. Agents such as drugs, environmental pollutants, food additives, tobacco or alcohol act as enzyme inducers (Pelkonen et al 1998). A “classical” definition of induction involves synthesis de novo of the enzyme as a result of an increased transcription of the corresponding gene, as a response to an appropriate stimulus. However, in studies on xenobiotic metabolism this term is often used in a wider sense to describe an increase in the amount and/or activity of the enzyme due to the action of chemical agents, regardless of the mechanism causing it (such as increased transcription, stabilisation of mRNA, increased translation or stabilisation of the enzyme) (Lin and Lu 1998). The phenomenon of induction is not exclusive of the CYP isoenzymes and also affects conjugation enzymes. However, the induction processes that have been studied in greater depth are those affecting the CYP isoenzymes and the inducers are classified according to the CYP isoenzymes on which they can act (Pelkonen et al 1998, Lin and Lu 1998).

However, not all of these differences in the biotransformation activity can be attributed to the action of inducers. It has been verified that genetic factors, specifically gene polymorphisms, are also involved in this variability (Smith et al 1998). CYP isoenzymes (CYP1A1/2, 2A6, 2C9, 2C19, 2D6, 2E1) and conjugation enzymes (N-acetyltransferase and glutathione S-transferase) are polymorphically expressed (Blum 1991, Miller et al 1997).

The gene polymorphism of P450, together with phenotypic variability, is the leading cause for interindividual differences in drug metabolism. This is due to the existence of genetic changes as a consequence of mutations, deletions and/or amplifications. Typically, there are two situations (Meyer and Zanger 1997): (i) subjects with defective genes (mutated, incomplete, non-existent, etc.) resulting in poor drug metabolisation (slow metabolisers); and (ii) individuals with duplicated or amplified functional genes which thus show a greater metabolisation capacity (ultrafast metabolisers).

The most widely studied polymorphisms are those of debrisoquine/sparteine hydroxylase (CYP2D6) (Skoda 1988; Kimura et al. 1989; Heim and Meyer 1992), and S-mephenytoin hydroxylase (CYP2C19) (Wrighton et al. 1993; De Morais 1994; Goldstein et al 1994), which respectively affect over 7% and 5% of the Caucasian population, and which can produce significant alterations in the metabolisation of over 30 commonly-used drugs.

Drug metabolism by hepatic enzymes must be understood as a set of reactions in which various enzymes compete for a same substrate, i.e., clinical relevance of metabolic variability and idiosyncrasy for the drug. The affinity of the drug for each enzyme (K_{M}) and the kinetic characteristics of the reaction catalysed by it (V_{MAX}) will determine the importance of the reaction in the overall context of the drug metabolism. Thus, two extreme situations may exist: a) the compound is a substrate for various enzymes, yet originates basically one metabolite, or b) several enzymes are involved in its metabolism, resulting in various metabolites being produced.

In the first case, a different expression of the enzymes involved in the metabolism of a drug results in differences in its rate of metabolisation, and thus in its pharmacokinetics. This phenomenon can result on one hand in a deficient drug metabolisation, with the ensuing accumulation of the compound in the organism, abnormally high plasma levels and, on the other hand, in a metabolisation so accelerated that it is impossible to attain suitable therapeutic levels and the desired pharmacological effect.

In the second case, the metabolic profile of the drug will be clearly different; that is, the amount and relative proportion of the metabolites produced would be different. This can translate into a lower pharmacological effectiveness if the metabolite, and not the compound administered, is pharmacologically active, or if abnormal amounts of a more toxic metabolite responsible for adverse effects are produced.

The geno-phenotypic variability of CYP isoenzymes, in addition to being directly responsible for the pharmacokinetic differences (bioavailability, half-life, rate and extent of metabolisation, metabolic profile) and indirectly responsible for the pharmacodynamic differences (therapeutic ineffectiveness/exaggerated response, undesired effects) (Miller et al 1997, Smith et al 1998), lies at the root of idiosyncratic toxicity (Pain 1995). Oftentimes, during its metabolism the drug can give rise to another metabolite more toxic to the cell, or be converted into a more reactive chemical species that can interact with other biomolecules (bioactivation). These type of reactions, a relative exception for a substantial part of the population, can have a considerable importance in other individuals with unusual expression levels of the various CYP isoenzymes (Meyer 1992).

Models predict effects due to changes in CYP isoenzyme expression. The availability of in vitro systems that can faithfully reproduce the in vivo metabolism of drugs is one of the goals pursued by various research groups. The research group of the inventors has developed cultivation of human hepatocytes and their use in pharmaco-toxicologic studies (Bort et al 1996, Castell et al. 1997, Gómez-Lechón et al 1997). However, in these models it is only possible to affect the expression of biotransformation enzymes to a limited extent. For example, using enzymatic inducers it is possible to increase the expression levels of CYP isoenzymes (Donato et al. 1995, Guillén et al. 1998, Li 1997). However, even using specific inducers such as methyl cholantrene, phenobarbital or rifampicin it is not possible to selectively modify one of them without affecting the others.

Another possible alternative is the use of genetically modified cell lines to overexpress one of the human CYP isoenzymes (Bort et al. 1999a). While these lines are a useful tool in determining whether a specific enzyme is involved in the formation of a given compound, they do not allow discovering the extent to which differences in expression of a biotransformation enzyme affect a drug's metabolic profile and rate of metabolisation by hepatocytes.

### SUMMARY OF THE INVENTION

The invention relates to obtaining a cell model capable of reproducing in vitro the metabolic idiosyncrasy of humans by expression vectors that encode for the sense and anti-sense mRNA of the Phase I and II drug biotransformation enzymes showing greatest variability in humans. This approach, based in the use of viral expression vectors, allows also to confer to any cell type (tumoral or not), of any tissue origin, the ability to express Phase I and/or Phase II biotransformation enzymes with activity against xenobiotics. When the biotransformation enzymes are CYP enzymes, it is necessary that, in addition, cells to be transfected show or express enough cytochrome P450 reductase activity to allow a suitable enzymatic activity. In general, cytochrome reductase expression levels in most primary cells are sufficient to allow a suitable enzymatic activity in cells transformed with the vectors herein described. However, if a cell line to be transformed by the inclusion of any sequence coding for a CYP enzyme does not show enough reductase activity, it can be co-infected simultaneously with two adenoviral vectors, the first one carrying the CYP sequence of interest, and the second one carrying the sequence of a CYP reductase, so that the cell line could express both enzymes. Alternatively, both genes could be included in the same adenoviral construct in order to infect the cells with both genes at the same time.

### BRIEF DESCRIPTION OF THE FIGURES

FIG. 1 illustrates the blocking of the expression of HNF4 by anti-sense RNA and repression of CYP2E1.

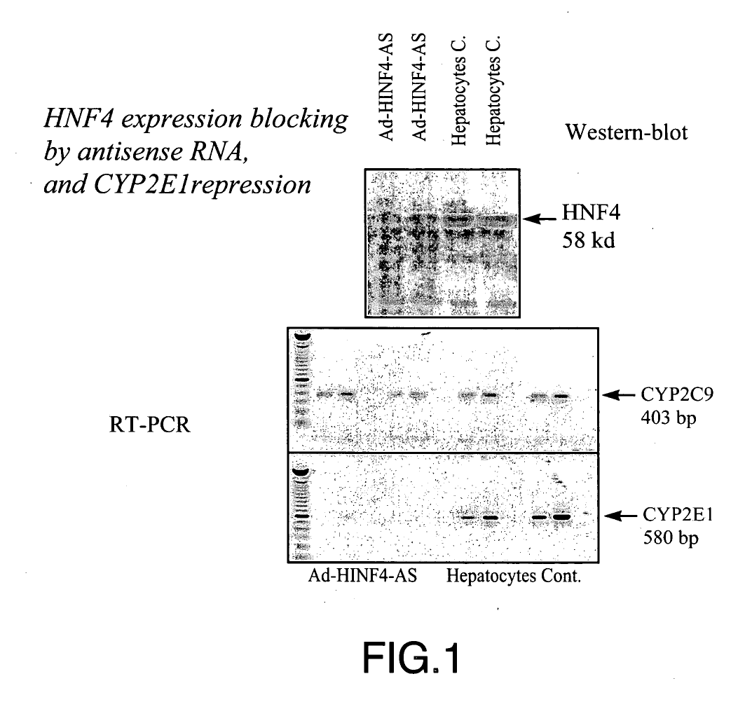

FIG. 2 is a bar chart showing the mRNA increase in HepG2I cells infected with different clones of the recombinant adenovirus identified as Ad-2E1.

FIG. 3 is a graph showing the increased activity in HepG2I cells infected with various concentrations of the recombinant adenovirus identified as Ad-3A4 and incubated with testosterone.

### DETAILED DESCRIPTION

The ideal model would be one allowing to modulate in a simple manner the individualised expression of an enzyme without affecting the others. In the case of induction, there are several experimental strategies that could be applied, based on the use of expression vectors with a promoter that can be activated by a specific exogenous compound in a concentration-dependent manner. In this way, depending on the activator concentration there will be a greater or lesser expression of the heterologous gene cloned “in phase” after the promoter.

A method for obtaining a cell model that can reproduce the metabolic idiosyncrasy of humans in vitro is disclosed. This method is based on the use of expression vectors that code for the sense and anti-sense mRNA of the drug biotransformation Phases I and II enzymes. These expression vectors preferably contain ectopic DNA sequences that code for the sense and anti-sense mRNA of drug biotransformation Phases I and II enzymes that present the greatest variability in humans.

The method allows modulating or modifying (increasing or diminishing) the individualised expression of an enzyme in a simple manner without affecting other enzymes. A cell model such as this one can be used in drug development studies, specifically in the study of drug metabolism, potential idiosyncratic hepatotoxicity, medicament interactions, etc.

A kit comprising one or more expression vectors that code for the sense and anti-sense mRNA of the enzymes of drug biotransformation Phases I and II is described that can be used to carry out the method for obtaining a cell model capable of reproducing in vitro the metabolic idiosyncrasy of humans.

A method for obtaining a cell model capable of reproducing in vitro the metabolic idiosyncrasy of humans, wherein said model comprises a set of expression vectors that confer to the transformed cells a phenotypic profile of drug biotransformation enzymes designed to reproduce the metabolic idiosyncrasy of humans, is disclosed,
a) Transforming cells expressing reductase activity with a set of expression vectors comprising ectopic DNA sequences that code for drug biotransformation enzymes selected from Phase I drug biotransformation enzymes and Phase II drug biotransformation enzymes,
   wherein each expression vector comprises an ectopic DNA sequence that codes for a different Phase I or Phase II drug biotransformation enzyme selected from:
   (i) A DNA sequence transcribed in the sense mRNA of a Phase I or Phase II drug biotransformation enzyme (sense vector) and
   (ii) a DNA sequence transcribed in the anti-sense mRNA of a Phase I or Phase II drug biotransformation enzyme (anti-sense vector);
   wherein the expression of said ectopic DNA sequences in the cells transformed with said expression vectors confers to the transformed cells certain phenotypic profiles of the Phase I or Phase II drug biotransformation enzymes, to obtain with said expression vectors cells that transiently express such DNA sequences and present a different phenotypic profile of Phase I or Phase II drug biotransformation enzymes; and
b) building a cell model capable of reproducing in vitro the metabolic idiosyncrasy of humans from said transformed cells transformed with said set of expression vectors, both sense vectors and anti-sense vectors, so that the result is the expression of any phenotypic profile of Phase I or Phase II drug biotransformation enzyme desired.

According to this method, cells that express reductase activity are transformed using a set of expression vectors. The existence of this reductase activity, CYP-reductase, in the cells to be transformed is essential, as if it is not present or is insufficient, the CYP protein contained in the expression vector will be expressed, but although it is active it will not be able to participate in the drug oxidation reactions.

The NADPH-cytochrome P450 reductase activity can be easily measured in the cells by an assay comprising, for example, cultivating the cells in 3.5 cm plates and using them when they reach 80% confluence. The cells are detached from the plates with the aid of a spatula in 1 ml of 20 mM phosphate buffer solution (PBS, pH 7.4), they are sonicated for 10-20 seconds and the homogenate obtained is centrifuged at 9,000 g for 20 minutes at 4° C. The supernatant (S-9 fraction) is used to evaluate the enzymatic activity. For this a 50 μl aliquot of the S-9 fraction protein is taken and incubated in 1 ml of 0.1 M potassium phosphate buffer (pH 7.2) containing 0.1 μM EDTA, 50 μM potassium cyanide, 0.05 μM cytochrome c and 0.1 μM NADPH. The reduction rate of the cytochrome c is determined by a spectrophotometer at 550 nm. The enzymatic activity is calculated using the molar extinction coefficient of 20×10³ M×cm⁻¹, and the results are expressed as nmol of cytochrome c reduced per minute and per mg of cell protein.

Practically any cell expressing reductase activity can be used to carry out this method, such as a human or animal cell, including tumour cells. Preferably, said cell is a human cell selected from among cells of hepatic, epithelial, endothelial and gastrointestinal type CaCO-2 origin. In one embodiment, this human cell is a hepatocyte or a HepG2I cell. In another embodiment, the cell expressing reductase activity is a human or animal cell, including tumour cells which, lacking the Phase I or Phase II drug biotransformation enzyme, is infected with a combination of one or more of the expression vectors, containing each of these in a certain concentration so that a cell is generated with a metabolic capability similar, for example, to that of a hepatocyte, with a normal or abnormal phenotype.

The expression vectors used to transform these cells expressing reductase activity, comprise the ectopic DNA sequences coding for drug biotransformation enzymes selected from among the Phase I drug biotransformation enzymes and Phase II drug biotransformation enzymes. Illustrative examples of Phase I and Phase II drug biotransformation enzymes include various oxygenases, oxidases, hydrolases and conjugation enzymes, among which the monooxygenases dependent on CYP450, flavin-monooxygenases, sulfo-transferases, cytochrome C reductase, UDP-glucuronyl transferase, epoxide hydrolase and glutathione transferase are enzymes greatly involved in drug biotransformation.

In general, each expression vector comprises an ectopic DNA sequence that codes for a different Phase I or Phase II drug biotransformation enzyme, selected from among the above-defined sequences (i) (sense) and (ii) (anti-sense).

Any DNA sequence coding for a Phase I or Phase II drug biotransformation enzyme can be used to build the expression vectors. However, in a specific embodiment the DNA sequence coding for a Phase I or Phase II drug biotransformation enzyme may be selected from DNA sequences transcribed in the sense mRNA or anti-sense mRNA of CYP450 isoenzymes, such as CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5 or DNA sequences transcribed in the sense mRNA or anti-sense mRNA of enzymes such as oxygenases, oxidases, hydrolases and conjugation enzymes involved in drug biotransformation, such as DNA sequences transcribed in the sense mRNA or anti-sense mRNA of the monooxygenases dependent on CYP450, flavin-monooxygenases, sulfo-transferases, cytochrome C reductase, UDP-glucuronyl transferase, epoxide hydrolase or glutathione transferase. The expression of these ectopic DNA sequences in the transformed cells confers to said cells certain phenotypic profiles of Phase I or Phase II drug biotransformation enzymes.

In one embodiment, said ectopic DNA sequence coding for a Phase I or Phase II drug biotransformation enzyme is a DNA sequence transcribed in the sense mRNA of a Phase I or Phase II drug biotransformation enzyme.

In another embodiment, the DNA sequence coding for a Phase I or Phase II drug biotransformation enzyme is a DNA sequence transcribed in the anti-sense mRNA of a Phase I or Phase II drug biotransformation enzyme.

The gene expression regulation strategy using anti-sense technology mainly consists of inserting in a cell an RNA molecule or an oligodeoxynucleotide whose sequence is complementary to that of a native mRNA that one desires to block. The specific and selective bonding of these molecules prevents translation of the messenger and synthesis of the corresponding protein (Melton 1985, Stein and Cheng 1993, Branch 1998). The final result is the targeted inactivation of the expression of a selected gene. The success of this strategy depends on various factors that are technically difficult to achieve, such as having an efficient system to insert the anti-sense molecule in the cell interior, said molecule interacting specifically with the target mRNA and not with other mRNA's, and that it is resistant to cell degradation systems. The two most commonly used procedures involve the use of an expression vector that includes a cloned cDNA in a reversed position (Melton 1995); when this vector is transfected into the cell interior it expresses a non-coding RNA or RNA fragment (nonsense RNA) that will associate by specific base pairing with its complementary native mRNA, or instead the use of oligo phosphothioates that are oligodeoxynucleotides modified to make them resistant to intracellular degradation (Stein and Cheng 1993). Its entry in the cell interior is solved by endocytosis or pinocytosis. The specific union to the target mRNA is harder to predict, so that the ideal oligo to block a specific mRNA can only be empirically determined [the success of this methodology has been greatly limited by the very low efficiency of the usual transfection procedures (10%)].

In one embodiment of the method, recombinant adenoviruses have been built that can be used as carriers of a cDNA cloned with an inverted orientation as a source of antisense mRNA inside the cell. As the transfection efficiency is very high, about 100%, the “antisense” molecule is expressed in a very efficient manner in almost all target cells. The simplicity of the infection process in hepatocytes, which are very resistant to classical transfection techniques, makes this the model of choice. The viability of the proposed strategy is supported by recent results obtained by the inventors developing an adenovirus that codes for the anti-sense mRNA of the hepatic transcription factor HNF4. Transfection of human hepatocytes with this anti-sense adenovirus results in the complete disappearance of the transcription factor HNF4 after 72 hours, as shown by western-blot analysis. The protein most homologous to HNF4 is another transcription factor of the same family known as RXRα. This protein does not undergo changes, thereby showing that the anti-sense blocking is highly specific. The targeted inactivation of this transcription factor led to the loss of expression of certain CYP's, specifically CYP2E1.

Almost any system for transferring exogenous DNA into a cell can be used to build the expression vectors. In a specific embodiment, the expression vector could be selected from, for example, among a viral vector, a liposome or a micellar vehicle, such as a liposome or micellar vehicle useful for gene therapy. In general, any virus or viral vector capable of infecting the cells used to put in practice the method can be used to build the expression vector. Advantageously, expression vectors will be chosen that can express transgenes in a highly efficient and quick manner in the transformed cells. In a specific embodiment, this virus is a natural or recombinant adenovirus, or a variant of it, such as a type 5 subgroup C adenovirus.

The adenovirus is a non-oncogenic virus of the Mastadenoviridae genus, whose genetic information consists of a double linear DNA chain of 36 kilobases (kb) divided into 100 mu (map units; 1 mu=360 bp). Information on its replicative cycle has been provided by Greber 1993, Ginsberg 1984 and Grand 1987.

The adenovirus easily infects many cell types, including hepatocytes, so that they are a useful tool for transfecting exogenous genes to mammal cells. Specifically, the adenovirus is an excellent expression vector that has the additional advantage of showing a very high efficiency for hepatocyte transfection (equal to or greater than 95%). Additionally, the expression degree is proportional to the infective viral load and, finally, the transgene expression does not affect the expression of other hepatic genes (Castell et al. 1998).

Introduction of ectopic genes in the DNA of an adenovirus is limited by two facts: (i) the virus cannot encapsulate more than 38 kb (Jones 1978 and Ghosh Choudhury 1987); and (ii) its large size hinders cloning as unique restriction points are infrequent. To solve these problems, several strategies have been employed, the most widely used of which is that developed by McGrory et al. 1988, homologous recombination. In short, the procedure essentially consists of using two plasmids, pJM17 and pACCMV, which contain a homologous fragment of the incomplete adenovirus sequence. Its homologous nature allows the recombination of the two plasmids, resulting in a defective (non replicative) virus in whose genome is the gene that must be expressed. Plasmid pJM17, developed by McGrory et al. 1988, is a large plasmid (40.3 kb) that contains the complete circularized genome of the type 5 adenovirus dl309 (Jones 1978) which has the plasmid pBRX (ori, ampr and tetr) in its locus XbaI in 3.7 mu. Although pJM17 contains all the necessary information for generating infective viruses, its size exceeds the encapsulation size so that it cannot generate new virions. In order for the adenovirus generated after recombination to be capable of reproducing, co-transfection is performed in the human embryonic cell line of renal origin 293 (ATCC CRL 1573) that expresses the region E1A of the type 5 adenovirus (Graham 1977). In this way, the supply of the protein E1A, a transcription factor acting in trans, by the host cell allows multiplying the recombinant virus inside it. It must be remarked that for its replication in the line 293 the recombinant virus also needs certain subregions of E1 in cys. These are the subregion lying between 0 and 1.3 mu, and that between 9.7 mu and the end of E1. Between 0 and 0.28 mu is the ITR (internal terminal repeats) with the replication origin, between 0.54 and 0.83 the packing signals (Hearing 1987) and lastly, after 9.7 mu, is a segment surrounding the gene of protein IX. For this reason these regions are maintained in pACCMV, in which only 3 kb have been eliminated from the E1 region to make room for the expression module, without preventing the normal replication of the virus in 293.

Example 1 shows how to obtain recombinant adenoviruses containing ectopic DNA sequences that are transcribed in the sense mRNA or antisense mRNA of CYP450 isoenzymes, such as CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5 or GST(A1). These recombinant adenoviruses can be used to transform (infect or transfect) cells expressing reductase activity, for example, cells of hepatic origin such as HepG2I.

One characteristic of the method lies in its versatility for generating cell models with specific phenotypes by only varying the concentrations of the expression vectors used to transform said cells. In fact, it is possible to obtain models that allow comparing the metabolism of a drug in a liver with 10 CYP3A4 and 1 CYP2D6 with respect to another with 1 CYP3A4 and 10 CYP2D6, for example, by simply changing the types and amounts of expression vectors to be used to transform the cells. Tests conducted by the inventors have revealed that the response of this model is practically linear, this is, the greater the amount of expression vector the more activity is expressed, up to a limit (when cytopathic effects appear in the cells). Several tests have revealed that, depending on the expression vector used, up to about 300 CFU (colony forming units) there are no significant alterations in any other function of the cells (human hepatocytes) transformed by said vectors.

Transformation of the cells with the expression vector can be performed by any conventional method for transferring DNA exogenous to a cell, such as infection or transfection, depending, among other factors, on the expression vector employed. In a specific embodiment, the expression vectors used are recombinant adenoviruses and the cells can be transformed by infection, for which the cells must be at 70% confluence. In short, the culture medium maintaining the cells is aspirated and the latter are washed with a base medium or saline buffer; two washes of 2 or 3 ml each shall be performed. The amount of virus to be used may vary, according to the amount of activity desired to be expressed by the cells and their susceptibility. The adenovirus is diluted in the culture medium until the concentration reaches the range of 1 to 50 MOI (multiplicity of infection). The volume of culture used to maintain the cells will depend on the size of the plate, the final infection volume will be reduced to ¼ of the initial volume. The incubation time will be between 1 hour 30 minutes and 2 hours, at 37° C. The activity of the transgene in the infected cells can be detected after 24 hours, reaching a maximum after 48 hours, depending on the cells used. The total maximum amount of virus that a specific cell will admit is limited. This amount is determined by adding increasingly large amounts of virus until apparent cytotoxic effects are observed (morphology, cell function). This allows establishing the maximum number of viral particles that a specific cell will tolerate.

The expression vectors can be used to transform transiently the cells expressing reductase activity. This transient transformation will be designed a priori to obtain the desired balance of expression of Phase I and Phase II drug biotransformation enzyme, in order to limit individual variability (metabolic idiosyncrasy), especially marked in the CYP system of humans. The combined use of variable amounts of different expression vectors (for example, some could express a Phase I or Phase II drug biotransformation enzyme and others their anti-sense mRNA) permits the necessary modulation, being established a priori, taking as a limit the viral load tolerated by each cell system.

Therefore, expression vectors, both sense and anti-sense, are used in a controlled manner, to modulate (increase or decrease) each of the Phase I or Phase II drug biotransformation enzymes in cells expressing reductase activity transformed by said vectors, so that these cells can reproduce a specific phenotype and provide an in vitro model for any conceivable human phenotypic profile, by adding a controlled amount of expression vector to said cells.

A considerable share of the problems arising in drug use (unexpected and undesirable effects, lack of or excessive therapeutic activity for the same dose, etc.) are due to the fact that humans do not metabolise drugs identically. Thus, the same dose can lead to different plasma levels in different individuals, and/or metabolise to give a different metabolite profile in different persons. It is often the case that because of the greater or lesser presence of a specific biotransformation enzyme, the hepatic metabolites produced (or their relative proportion) can be quite different. Occasionally, low levels of enzymes, whose action results in production of low toxicity metabolite(s), is poorly expressed in a given individual, so that metabolism of the drug in this individual will follow alternative paths that may produce much more toxic metabolites which are a minority in other individuals. In other cases it can be the abnormally high presence of a given enzyme, in the minority in other individuals, that leads to the production of a more toxic metabolite. These differences (metabolic idiosyncrasy) are an added risk factor in the arduous task of developing a molecule into a new drug. The reason for this is simple: compounds that have not shown adverse effects in the first clinical assays may, when widening their use to a greater population, allowing entry of individuals with metabolic abnormalities, produce idiosyncratic toxicity effects that can cause the failure of the compound.

Deliberate manipulation of the levels of the various drug biotransformation enzymes of a human cell, as occurs in humans, permits study in the cell whether the changed levels can be relevant in a generalised clinical use of a new compound.

Therefore, in another aspect, expression vectors (sense or anti-sense) of Phase I or Phase II drug biotransformation enzymes are used in the manipulation of cells, such as human and animal cells, including tumour cells, in order to reproduce in these cells the metabolic variability occurring in humans. Said vectors allow modifying at will the expression of a given enzyme without affecting the others. In this way it is possible to manipulate cells, making them express the amounts of each enzyme desired (as viral vectors can be used alone or in combination), thereby simulating the variability that occurs in humans. Thus, the possible relevance for a person of different expression levels of drug biotransformation enzyme when administering a new drug, can be studied and anticipated before the drug is used in humans, thereby constituting an experimental cell model allowing one to simulate or reproduce in vitro the variability existing in humans. In addition, consequences of the different expression of drug biotransformation enzymes on the metabolism, pharmacokinetics and potential hepatotoxicity of a drug in process of development can be predicted.

In another aspect, a kit comprising one or more expression vectors coding for the sense and anti-sense mRNA of Phase I and Phase II drug biotransformation enzymes is described. This kit can be used to put in practice the method for obtaining a cell model capable of reproducing in vitro the metabolic idiosyncrasy of humans.

### EXAMPLE 1

### Generation of Recombinant Adenoviruses

Cloning of Various Human Biotransformation Enzymes from an Own Human Liver Bank

The strategy used for cloning human CYP biotransformation enzymes 1A1, CYP 1A2, CYP 2A6, CYP 2B6, CYP 2C8, CYP 2C9, CYP 2C18, CYP 2C19, CYP 2E1, CYP 3A4, CYP 3A5 or GST(A1) was performing a high-fidelity RT-PCR on a library of human hepatic cDNA's using primer oligonucleotides that flank the sequences coding for such enzymes.

The reaction mixture for reverse transcriptase (RT) consisted of 20 μl 1× reverse transcriptase buffer, DTT 10 mM, dNTPs 500 μM, 3 μM primer oligo d(T), 14, 60 U Rnase OUT and 250 U Rtase H. To this mixture was added 1 μg of total RNA. The reaction was performed for 60 minutes at 42° C, followed by heating for 5 minutes at 95° C. and a quick cooling in ice. The cDNA was stored at −20° C. until it was used.

Primer Oligonucleotides Used

For each CYP two pairs of primer oligonucleotides flanking their coding sequence were designed. Each primer contains an additional sequence in the 5′ end corresponding to a restriction site for a specific enzyme, wherein they will be cloned in the pACCMV vector [see Table 1].

TABLE 1
Primer oligonucleotides used to clone the genes

| | | | | |
|---|---|---|---|---|
| | | Fragments | Melting | Page |
| Oligonucleotides | Sequences 5′ to 3′ | (pb) | T (° C.) | no. |

| | | | | |
|---|---|---|---|---|
| CYP 1A1 FP | cctccaggatccctacactgatc | | | |
| | (SEQ ID NO. 1) | | | |
| CYP 1A1 RP | cccggatcccagatagcaaaac | | | |
| | (SEQ ID NO. 2) | | | |
| CYP 1A2 FP | gcaggtaccgttggtaaagatggcatt | 1596 | 62.0 | M14337 |
| | (SEQ ID NO. 3) | | | |
| CYP 1A2 RP | agccatggaccggagtcttaccaccac | | 60.8 | |
| | (SEQ ID NO. 4) | | | |
| CYP 2A6 FP | cccgaattcaccatgctggcctcagg | 1531 | 64.0 | X13930 |
| | (SEQ ID NO. 5) | | | |
| CYP 2A6 RP | cgaattccagacctgcaccggcaca | | | |
| | (SEQ ID NO. 6) | | | |
| CYP 2B6 FP | cagggatcccagaccaggaccatggaa | 1482 | 62.7 | M29874 |
| | (SEQ ID NO. 7) | | | |
| CYP 2B6 RP | tttgggatccttccctcagccccttcag | | | |
| | (SEQ ID NO. 8) | | | |
| CYP 2C8 FP | ggggtaccttcaatggaaccttttgtgg | 1515 | | Y00498 |
| | (SEQ ID NO. 9) | | | |
| CYP 2C8 RP | cccaagcttgcattcttcagacaggg | | | |
| | (SEQ ID NO. 10) | | | |
| CYP 2C9 RP | ggaattcggcttcaatggattctcttgtgg | 1485 | | M61855 |
| | (SEQ ID NO. 11) | | | |
| CYP 2C9 FP | cgtctagacttcttcagacaggaatgaa | | | |
| | (SEQ ID NO. 12) | | | |
| CYP 2C18 FP | cccgaattcaccatgctggcctcagg | 1515 | | M61853 |
| | (SEQ ID NO. 13) | | | |
| CYP 2C18 RP | ccgaattccagacctgcaccggcaca | | | |
| | (SEQ ID NO. 14) | | | |
| CYP 2C19 FP | atggatccttttgtggtcctt | | | M61854 |
| | (SEQ ID NO. 15) | | | |
| CYP 2C19 RP | agcagccagaccatctgtg | | | |
| | (SEQ ID NO. 16) | | | |
| CYP 2D6 FP | ctaagggaacgacactcatcac | | | |
| | (SEQ ID NO. 17) | | | |
| CYP 2D6 RP | ctcaccaggaaagcaaagacac | | | |
| | (SEQ ID NO. 18) | | | |
| CYP 2E1 FP | | 1649 | | J02625 |
| CYP 2E1 RP | | | | |
| CYP 3A4 FP | | 1602 | | M18907 |
| CYP 3A4 RP | | | | |
| CYP 3A5 FP | gttgaagaatccaagtggcgatggac | 1707 | 58.3 | J04813 |
| | (SEQ ID NO. 19) | | | |
| CYP 3A5 RP | acagaatccttgaagaccaaagtagaa | | 53.0 | |
| | (SEQ ID NO. 20) | | | |
| GST(A1) FP | ccaggatcctgctatcatggcagagaa | 735 | 50.9 | M21758 |
| | (SEQ ID NO. 21) | | | |
| GST(A1) RP | tatggatcccaaaactttagaacattggtattg | | 47.9 | |
| | (SEQ ID NO. 22) | | | |

High Fidelity PCR

The newly synthesised cDNA is used to conduct a conventional PCR. The PCR reaction was conducted in a thermocycler with the following reaction mixture: 3 μl of cDNA (1/10 RT), 3 μl buffer (10×), 5 μM dNTPs, 1 U total High Fidelity (Roche), 6 μM primer oligonucleotides and water to a final volume of 30 μl. The program used in the thermocycler consisted of:
A) Initial denaturation: 3 minutes at 95° C.
B) 4 cycles of:
a.—denaturation by cycles: 40 s at 95° C.
b.—amplifying: 45 s at 58° C. (different for each primer)
c.—final elongation: 5 minutes at 74° C.
C) 30 cycles (more specific) of:
a.—denaturation by cycles: 40 s at 95° C.
b.—amplifying 45 s at 62° C. (different for each primer)
c.—followed by a final elongation of 5 minutes at 74° C.

The product amplified by PCR was purified by column chromatography (High pure PCR product purification kit) and eluted by TE buffer. Then the PCR products were analysed by electrophoresis in 1.5% agarose gel and visualised with ethidium bromide to confirm the sizes of the amplified cDNA's.

Characterisation of the Cloned Genes. Digestions With Restriction Enzymes. Agarose Gels. Sequencing

Prior to cloning the DNA was incubated with restriction enzymes in the buffer recommended by the manufacturer. A standard incubation mixture includes: 2 units of enzyme/pg of DNA, 10× buffer and distilled water. Occasionally, some enzymes require 100 μg/ml BSA or are incubated at 25° C.

Generation of PA CCCMV Recombinant Plasmids

Subcloning of cDNA fragments (insert) in a pACCMV vector (vector) was performed by ligation of cohesive ends with the same restriction enzyme. This strategy produces clones with a sense and anti-sense orientation. In addition to the ligation itself, it includes prior dephosphorylation steps of the vector ends to prevent their recircularisation, for which added to the previous tube were 2 μl of CIP (20-30 U/μl; Gibco BRL cat n° 18009019) and it was incubated for 20 minutes at 37° C. Then another 2 μl of CIP are added and it was incubated for 20 minutes at 56° C. To inactivate the enzyme and stop the reaction it was incubated for 10 minutes at 75° C.

Before ligation, the vector and the insert must be purified to eliminate remains of nucleotides, enzymes and buffers that may hinder the ligation. For this, the Geneaclean kit (Bio 101 cat n° 1001 -200) is used to purify bands of a TAE-agarose gel (1% agarose in Tris-acetate 40 mM and EDTA 2 mM).

After purifying both bands the following reaction mixture was prepared for ligation:
2 μl vector (0.75 μg/μl)
4 μl insert (1 μg/μl)
1 μl T4 Ligase (1 U/μl) (Gibco BRL cat n° 15224-017)
1.5 μl 10× buffer
6.5 μl water
15.0 μl total

In parallel, a control mixture without insert was prepared. After 2 hours at ambient temperature competing bacteria were transformed with the ligation mixtures.

Ligation of cohesive ends was performed with the following reaction mixture:
b 1 μl vector (0.5 μg/μl)
4 μl insert (1 μg/μl)
1 μl T4 Ligase (1U/μl) (Gibco BRL cat n° 15224-017)
1.5 μl 10× buffer
10.0 μl water

In parallel, a control mixture without insert was prepared. After 2 hours at ambient temperature competing bacteria were transformed with the ligation mixtures.

Amplification of the Plasmids in Bacteria

Bacteria were used that had been previously treated with cold CaCl₂ solutions and subjected for a very short time to 42° C. to make them competent and receptive to the plasmid DNA: For this, 0.1-1 μg cDNA were added (ligation) to 100 μl of competent bacteria, the mixture was left in ice for 30 minutes and it was incubated in 1 ml of S.O.C. medium (Gibco BRL cat n° 15544-0189). Then 100 μl were transferred to an LB-agar medium plate with ampicillin (100 μg/ml) and it was left overnight at 37° C.

After this the bacteria were allowed to grow and they were used to amplify and purify the plasmid DNA by the procedure described hereinafter. An isolated colony of transformed bacteria is grown in 2-5 ml of LB medium with ampicillin. Then it is centrifuged at 8,000 rpm for 1 minute and the precipitate is resuspended in a lysis buffer (glucose 50 mM, Tris-HCl 25 mM, ph 8.0, EDTA and 4 mg/ml of lysozyme). The suspension is left on ice for 5 minutes and is centrifuged at 10,000 rpm for 5 minutes. The supernatant is transferred to a clean tube, 500 μl isopropanol is added and it is centrifuged at 15000 rpm for 10 minutes. The supernatant is removed and the residue is washed with 70% ethanol (v/v), dried and resuspended in a suitable volume of TE pH 7.5 (Tris 10 mM, EDTA 1 mM).

After verifying the colony with restriction enzymes, the rest of the culture is transferred to a flask with 250 ml and it is grown overnight to amplify the plasmid.

Conventional kits were used to purify the plasmid DNA of the bacteria culture (between 250 and 500 ml).

Generation of the Adenovirus. Co-Transfection of pJM17 and pA C-CYP Plasmids in 293 Cells

Co-transfection of the plasmids is performed in the 293 cell line, in which the recombinant virus generated by homologous recombination is able to replicate.

Co-transfection of the plasmids was performed by the calcium phosphate method, using different proportions. For this several plates of 6 cm diameter are seeded at 50-60% confluence. The next day tubes are prepared containing the different plasmids and/or carriers as well as the controls, and the content of each tube is added dropwise to 500 μl of HBS 2× (Hepes 50 mM, NaCl 140 mM, KCl 5 mM, glucose 10 mM and Na₂HPO₄ 1.4 mM adjusting to pH 7.15) and it is left for 20 minutes at ambient temperature. Then it is poured gently on the cell monolayer avoiding detachment, it is left for 15 minutes at ambient temperature, 4 ml of medium with serum are added, it is incubated in an oven at 37° C. for 4-6 hours, the medium is removed from the plates, 1 ml of medium without serum or antibiotics is added with 15% glycerol, 90 seconds are allowed to elapse and 5 ml PBS are added. Then it is washed twice with PBS to remove the glycerol completely, 5 ml of medium are added and it is stored in an oven, changing the medium every 3-4 days until cell lysis is observed.

After the recombination process occurs the virus will replicate in the 293 cells, managing to produce lysis in them (from 2 to 6 days). Then the virus is cloned, for which plates covered with semisolid agar with serial 1/10-1/100 dilutions of the virus to be cloned are prepared in DMEM and 0.5 ml of each dilution are added to a 6 cm diameter plate with 293 cells, and the cells are incubated in an oven at 37° C. for 1 hour, shaking them every 15 minutes. Then the medium is removed and the monolayer is covered with 6 ml of a mixture of agar 1.3% MEM 2× (1:1 v/v) previously heated to 45° C. and it is incubated in an oven at 37° C. After 7-9 days bald patches are visible, or areas in which the 293 cell monolayer is altered. These bald patches are selected and amplified in new plates of 293 cells.

Adenovirus Purification by Precipitation with PEG8000

A stock of pure virus was prepared by centrifugation in a CsCl gradient (method A) and, alternatively, using polyethylene glycol (method B), a simple method yielding similar results.

Method A

When the 293 cells undergo lysis the supernatant is removed and they are collected in PBS with MgCl₂ 1 mM, and 0.1% Nonidet p40.

Method B

In this case the cells have already undergone lysis and thus it is not possible to remove the medium. Nonidet p40 is added until it is left at 0.1%. It is then shaken for 10 minutes at ambient temperature and centrifuged at 20,000 g for 10 minutes. The supernatant is transferred to a clean tube and 0.5V are added of 20% PEG-8000/NaCl 2.5M, and it is incubated with shaking for 1 hour at 4° C. It is then centrifuged at 12,000 g for 10 minutes and the precipitate is resuspended in 1/100 to 1/50 of the initial medium volume in the following buffer: NaCl 135 mM, KCl 5 mM, MgCl₂ 1 mM and Tris-HCl 10 mM pH 7.4. Then it was dialysed overnight at 4° C. with the same buffer and filtered through a 0.22 μm filter to sterilise the stock. Finally, aliquots were obtained and conserved at −70° C. with 100 μg/ml de BSA.

Following the above procedure, recombinant adenoviruses were generated containing the DNA sequences coding for the CYP biotransformation enzymes CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5 or GST(A1). These recombinant adenoviruses (expression vectors) were named with the prefix “Ad” (adenovirus) followed by the name of the enzyme, this is, Ad-1A1, Ad-1A2, Ad-2A6, Ad-2B6, Ad-2C8, Ad-2C9, Ad-2C18, Ad-2C19, Ad-2D6, Ad-2E1, Ad-3A4, Ad-3A5 and Ad-GST(A1) respectively.

### EXAMPLE 2

### Transformation of Cells Expressing C Reductase Cytochrome Activity with Recombinant Adenoviruses

The recombinant adenoviruses obtained in Example 1 [Ad-1A1 Ad-1A2, Ad-2A6, Ad-2B6, Ad-2C8, Ad-2C9, Ad-2C18, Ad-2C19, Ad-2D6, Ad-3A5 and Ad-GST(A1)] were used to transform HepG2I cells by infection.

The culture medium containing a culture of HepG2I cells at 70% confluence was aspirated. The cells were washed twice with 2-3 ml of base medium or saline buffer each time. The amount of virus used varied widely in order to generate a cell model encompassing a wide spectrum of human metabolic variability. The adenoviruses were diluted in the culture medium until reaching a concentration from 1 to 50 MOI. The volume of medium used to maintain the cells depends on the plate size, the final infection volume will be reduced to ¼ of the usual volume. The incubation time was from 1 hour 30 minutes to 2 hours at 37° C. The activity of the transgene in the infected cells can be detected after 24 hours, reaching a maximum after 48 hours, depending on the cell used. The maximum amount of total viruses admitted by a given cell is limited. To determine this amount increasingly large amounts of virus are added until apparent cytotoxic effects (morphology, cell function) are observed. In this way it has been possible to establish the maximum number of viral particles tolerated by a given cell.

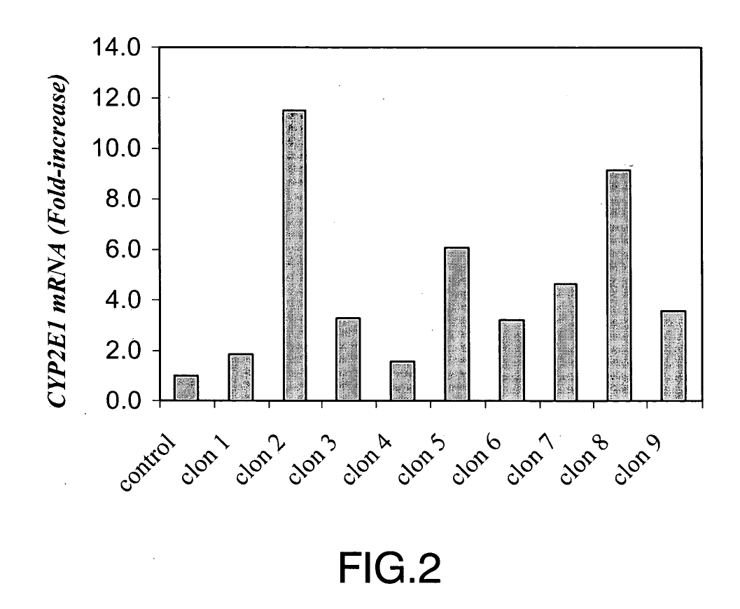

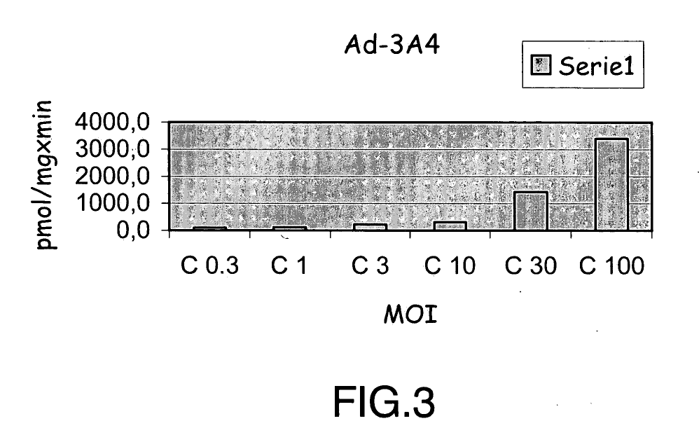

FIGS. 2 and 3 show specific examples of how it is possible to modify at will the expression of human enzymes relevant to drug metabolism. Specifically, FIG. 2 shows the increase of mRNA in HepG2I cells infected with various clones of Ad-2E1, while FIG. 3 shows the increased activity in HepG2I cells infected with various concentrations of Ad-3A4 and incubated with testosterone.

### BIBLIOGRAPHY

Andersson, T., Miners, J. O., Veronese, M. E., Tassaneeyakul, W., Tassaneeyakul, W., Meyer, U. A. and Birkett, D. J. (1993) Identification of human liver cytochrome P450 isoforms medianting omeprazole metabolism Br. J. Clin. Pharmacol. 36: 521-530.
Banks A. T., Zimmerman H. J. and Harter J. G. (1995) Diclofenac-associated hepatotoxicity: analysis of 180 cases reported in the Food and Drug Administration as adverse reactions. Hepatology 22: 849-871
Bort R, Ponsoda, X., Carrasco, E., G6mez-Lech6n M. J. and Castell, J. V. (1996) Metabolism of aceclofenac in humans. Drug. Met. Disp. 24: 834-851
Boelsterli U. A. (1993) Specific targets of covalent drug-protein interactions in hepatocytes and their toxicological significance in drug-induced liver injury. Drug Metaol. Rev 25: 395-451.
Bort, R., Mace, K., Boobis, A., Gómez-Lechón, M. J., Pfeifer, A. and Castell, J. V. (1999a), Hepatic metabolism of diclofenac. Role of human CYP in the minor oxidative pathways. Biochem. Pharmacol. en prensa.
Bort R., Ponsoda X., Jover, R., Gómez-Lechón, M. J. and Castell, J. V. (1999b) Diclofenac toxicity to hepatocytes: A role for drug metabolism in cell toxicity J. Pharmacol. Exp. Ther en prensa.
Blum, M., Demierre, A., Grant, D., Heim, M. and Meyer, U. (1991) Molecular mechanism of slow acetylation of drugs and carcinogens in humans. Proc. Natl. Acad. Sci. USA. 88: 5237-5241.
Branch A. D (1998) A good antisense molecule is hard to find. TIBS 23:45-50,
Castell, J. V., Hernandez, D., Gómez-Foix, A.M., Guillén, I., Donato, T. and Gómez-Lechón, M. J. (1997) Adenovirus-mediated gene transfer into human hepatocytes: analysis of the biochemical functionality of transduced cells. Gene Ther. 4: 455-464
Castell, J. V., Gómez-Lechón, M. J., Ponsoda, X. and Bort, R. (1997) The use of cultured hepatocytes to investigate the mechanisms of drug hepatotoxicity. Cell Biol. Toxicol. 13: 331-338.
Chardonnet, Y. and Dales, S. (1970) Early events in the interaction of adenoviruses with HeLa cells. I. Penetration of type 5 and intracellular release of the DNA genome. Virology 40: 462-477.
Christians, U., Schmidt, G., Bader, A., Lampen, A., Schottmann, R., Linck, A. and Sewing, K. F. (1996) Identification of drugs inhibiting the in vitro metabolism of tacrolimus by human liver microsomes. Br. J. Clin. Pharmacol. 41: 187-190.
De Morais, S. M. F., Wilkinson, G. R. and Blaisdell, J. (1994)The major genetic defect responsible for the polymorfism of S-mephenytoin metabolism in humans. J. Biol. Chem. 269: 15419-15422.
Defer, C., Belin, M. T., Caillet Boudin, M. L. and Boulanger, P. (1990). Human adenovirus host interaction: comparative study with members of subgroups B and C. J. Virol. 67: 3661-3673
Donato, M. T., Gómez-Lechón, M. J. and Castell, J. V. (1993) A microassay for measuring cytochrome P4401A1 and P45011B1 activities in intact human and rat hepatocytes cultured on 96-well plates. Anal. Biochem 213: 29-33.
Donato, M. T., Gómez-Lechón, M. J. and Castell, J V (1992) Biotransformation of drugs by cultured hepatocytes. En: In Vitro Alternatives to Animal Pharmaco-Toxicology. Eds J. V. Castell and M. J. Gómez-Lechón. Farmaindustria, Madrid, Chapter 7, pp. 149-178,
Donato M. T. , J. V. Castell and M. J. Gómez-Lechón (1995) Effect of model inducers on cytochrome P450 activities of human hepatocytes in primary cultures. Drug Metab Disp 23: 553-558
Donato, M. T., Castell, J. V. and Gómez-Lechón, M. J. (1998) The coumarin 7-hydroxylation microassay in living hepatic cells in culture. ATLA 26: 213-233.
Fitzgerald, D. J. P., Padmanabhan, R., Pastin, P. and Willingham, M. C. (1983) Adenovirus induced release of epidermal growth factor and Pseudomonas toxin into the cytosol of KB cells during receptor mediated endocytosis. Cell 32: 607-617
Furukori, H., Otani, K., Yasui, N., Kondo, T., Kaneko, S., Shimoyama, R., Ohkubo, T., Nagasaki, T. and Sugawara, K. (1998) Effect of carbamazepine on the single oral dose pharmacokinetics of alprazolam. Neuropsychopharmacology 18: 364-369.
Garattini, S. (1994) Perspectives in the field of drug metabolism. Drug Metab. Rev. 26: 537-73
Ghosh-Choudhury, G., Haj Ahmad, Y. and Graham, F. L. (1987) Protein IX, a minor component of the human adenovirus capsid, is essential for the packaging of full length genomes. EMBO J. 6: 1733-1739
Gil, M. L., Ramirez, M. C, Terencio, M. C. and Castell, J. V. (1995) Immunochemical detection of protein adducts in cultured human hepatocytes exposed to diclofenac. Biochim. Biophys. Acta 1272: 140-146
Ginsberg, H. S (1984). The adenoviruses. Plenum Press, New York.
Gluzman, Y., Reichl, H. and Solnick, D. (1982) Helper free adenovirus type 5 vectors. En Eucaryotic viral vectors (Y. Gluzman ed.) pp. 187-192. Cold Spring Harbor Laboratory Press, Cold Spring Harbor, New York.
Goldstein, J. A., Faletto, M. B., Romkes-Sparks, M., Sullivan, T., Kitareewan, S., Raucy, J. L., Lasker, J. M. and Ghanayem, B. I. (1994) Evidence that CYP2C19 is the major (S)-mephenytoin 4-hydroxylase in humans. Biochemistry 33: 1743-52.
Gómez-Foix, A. M., Coats, W. S., Baque, S., Alam, T., Gerard, R. D. and Newgard, C. B. (1992) Adenovirus mediated transfer of the muscle glycogen phosphorylase gene into hepatocytes confers altered regulation of glycogen metabolism. J. Biol. Chem. 267: 25129-25134.
Gómez-Lech\acute{0}n, M. J., Donato, T., Ponsoda, X., Fabra, R., Trullenque, R. and Castell, J. V. (1997) Isolation, culture and use of human hepatocytes in drug research. In In Vitro Methods in Pharmaceutical Research. J. V. Castell and M. J. Gómez-Lechón eds. pp. 129-154. Academic Press. London.
Gonzalez, F. J. and Gelboin, H. V. (1991) Human cytochromes P450: Evolution, catalytic activities and interindividual variations in expression. En: New Horizons in Biological Dosimetry. pp.11 20. Wiley Liss, Inc.
Gossen, M. and Bujark, H. (1992) Tight control of gene expression in mammalian cells by tetracycline-responsive promoters. Proc. Natl. Acad. Sci USA 89: 5547-5551.
Gossen, M., Freundlieb, S., Bender, G., Muller, G., Hillen, W. and Bujard, H. (1995) Transcripcional activation by tetracyclines in mammalian cells Science 268: 1766-1769.
Graham, F. L., Smiley, J., Russell, W. C. and Naim, R. (1977) Characteristics of a human cell line transformed by DNA from human adenovirus type 5. J. Gen. Virol. 36: 59-64.
Grand, R. J. The structure and functions of the adenovirus early region 1 proteins. Biochem. J. 241: 25-38, 1987.
Greber, U. F., Willets, M., Webster, P. and Helenius, A. (1993) Stepwise dismantling of adenovirus 2 during entry into cells. Cell 75: 477-486,
Guillen I., Donato, M. T., Jover, R., Castell, J. V., Fabra, R., Trullenque, R. and Gómez-Lechón, M. J. (1998) Oncostatin M down-regulates basal and induced cytochromes P450 in human hepatocytes. J. Pharm. Exp. Ther.285:127-134.
Guillouzo, A. and Chesne, C. (1996) Xenobiotic metabolism in epithelial cell cultures. En Cell culture models in epithelial tissues: A practical approach (Shaw A. J. ed.) pp. 67-85. Oxford, Oxford, U.K.
Hearing, P., Samulsky, R. J., Wishart, W. L. and Shenk (1987) Identification of a repeated sequence element required for efficient encapsulation of the adenovirus type 5 chromosome. J. Virol. 61: 2555-2558,
Heim, M. H. and Meyer, U. A. (1992) Evolution of a highly polymorphic human cytochrome P450 gene cluster: CYP2D6. Genomics 14: 49-58.
Helsby, N. A.; Ward, S. A.; Parslew, R. A. G.; Friedmann, P. S. and Rhodes, L. E. (1998) Hepatic cytochrome P450 CYP2C activity in psoriasis: studies using proaguanil as a probe compound. Acta Dermato Venereologica 78:81-83.
Jones, N. and Shenk, T. (1978) Isolation of deletion and substitution mutants of Adenovirus Type 5. Cell 13: 181-188.
Kimura, S., Umeno, M., Skoda, R. C., Meyer, U. A. and Gonzalez, F. J. (1989) The human debrisoquine 4-hydroxylase (CYP2D) locus: sequence and identification of the polymorphic CYP2D6 gene, a related gene, and a pseudogene. Am. J. Hum. Genet. 45:889-904,
Krishna, D. R. and Klotz, U. (1994) Extrahepatic metabolism of drugs in humans. Clin-Pharmacokinet 26:144-160.
Kronbach, T., Mathys, D., Gut, J., Catin, T. and Meyer, U. A. (1987) High-performance liquid chromatographic assays for bufuralol 1′-hydroxylase, debrisoquine 4-hydroxylase, and destromethorphan O-demethylase in microsomes and purified cytochrome P-450 isozymes of human liver. Analytical Biochemistry 162: 24-32
Lautenschlager, M. T., Viktor, S., Muller, U. A. and Hoffmann, A. (1996) Serum concentrations of caffeine, metamizol, ebrisoquine, sulfamethazine and their metabolites in diabetics before and during insulin therapy. Pharmazie 51: 750-753.
Lecoeur, S., Bonierbale, E., Challine, D., Gautier, J. C., Valadon, P., Dansette, P. M., Catinot, R., Ballet, R., Mansuy, D. and Beaune, P. H. (1994) Specificity of in vitro covalent binding of tienilic acid metabolites to human liver microsomes in relationship to the type of hepatotoxicity: comparison with two directly hepatotoxic drugs. Chem. Res. Toxicol. 7: 434 442.
Lewis, J. H. (1984) Hepatic toxicity of nonsteroidal anti-inflammatory drugs. Clin. Pharm. 3: 128-138
Li, A. P. (1997) Primary hepatocyte cultures as an in vitro experimental model for evaluation of pharmacokinetic drug-drug interactions. In Drug-Drug Interactions. Scientific and regulatory perspectives. A. P. Li, ed. pp. 103-130. Advances in Pharmacology, Academic Press. N.Y.
Lin, J. H. and Lu, Y. H. (1998) Inhibition and induction of cytochrome P450 and clinical implications. Clin. Pharmacokinet 35: 361-390.
McGrory, W. J., Bautista, D. and Graham, F. L. (1988) A simple technique for the rescue of early region I mutations into infectious human adenovirus type 5. Virology 163:614 617.
Meeks, R. G., Harrison S. D. and Bull R. J. (1991). Hepatotoxicology. (R. G. Meeks, S. D. Harrison and R. J. Bull, eds.). CRC Press, Boca Raton, Fla.
Melton, D. A. (1985) Injected anti-sense RNAs specifically block messenger RNA translation in vivo. Proc. Natl. Acad. Sci. USA 82:144-148.
Meyer, U. A. (1992) Drugs in special patient groups: Clinical importance of genetics in drug effects. In Clinical Pharmacology Basic Principles of Therapeutics. K. F. Melmon and H. F. Morelli, eds. Pp. 875-894. McGraw-Hill. N.Y.
Meyer, U. and Zanger, U. (1997) Molecular mechanisms of genetic polymorphisms of drug metabolism. Annu. Rev. Pharmacol. Toxicol 37: 269-96.
Miller, M. S., McCarver, D. G., Bell, D. A., Eaton, D. L. and Goldstein, J. A. (1997) Genetic polymorphisms in human drug metabolic enzymes. Fundam. Appl Toxicol 40:1-14.
Miners, J. O. and Birkett, D. J. (1996) The Use of Caffeine as a Metabolic Probe for Human Drug-Metabolizing-Enzymes. General Pharmacol. 27:245-249.
Miners, J. O., Coulter, S., Tukey, R. H, Veronese, M. E. and Birkett, D. J. (1996) Cytochrome P450, 1A2, and 2C9 are responsible for the human hepatic O-demethylation of R- and S-naproxen. Biochem. Pharmacol. 51: 1003-1008, No, D., Yao, T. P. and Evans, R. M. Ecdysone-inducible gene expression in mammalian cells and transgenic mice. Proc. Natl. Acad. Sci USA 93: 3346-335.
Ourlin, J. C., Vilarem, M. J., Daujat, M., Harricane, M. C., Domergue, J., Joyeux, H., Baulieux, J. and Maurel, P. (1997) Lipid-mediated transfection of normal adult human hepatocytes in primary culture. Anal. Biochem. 247: 34-44.
Pain, A. J. (1995) Heterogeneity of cytochrome P450 and its toxicological significance. Hum. Exp. Toxicol. 14: 1 7.
Park, B. K. and Kitteringham, N. R. (1990) Drug protein conjugation and its immunological consequences. Drug Metab. Rev. 22: 87 144.
Pearce, R. E., McIntyre, C. J., Madan, A., Sanzgiri, U., Draper, A. J., Bullock, P. L., Cook, D. C., Burton, L. A., Latham, C. N. and Parkinson A. (1996) Effects of freezing, thawing, and storing human liver microsomes on cytochrome P450 activity. Archives of Biochemistry and Biophysics 331: 145-169.
Pelkonen, O., Maenpaa, J., Taavitsainen, P., Rautio, A. and Raunio, H. (1998) Inhibition and induction of human cytochrome P450 (CYP) enzymes. Xenobiotica 28: 1203-1253.
Peter, R., Bocker, R., Beaune, P. H., Iwasaki, M., Guengerich, F. P. and Yang, C. S. (1990) Hydroxylation of chlorzoxazone as a specific probe for human liver cytochrome P-45011B1. Chem. Res. Toxicol. 3: 566-573.
Philipson, L., Lonberg, K. and Petterson, U. (1968) Virus receptor interaction in an adenovirus system. J. Virol. 2: 1064-1075.
Ponsoda, X., Bort, R., Jover, R., G\acute{0}mez-Lechón, M. J. and Castell, J. V. (1995) Molecular mechanisms of diclofenac hepatotoxicity: cell injury is associated to the metabolism of the drug and is preluded by a decrease in ATP levels. Toxicology in Vitro 9: 439-444.
Rahman, A., Korcekwa, R., Gorgan, J., Gonzalez, F. J. and Harris J. W. (1994) Selective biotransformation of Taxol to 6a-Hydroxytaxol by human cytochrome P450 2C8. Cancer Res. 54: 5543-5546.
Rasmussen, E., Eriksson, B., Oberg, K., Bondesson, U. and Rane, A. (1998) Selective effects of somatostatin analogs on human drug-metabolizing enzymes. Clin. Pharmacol. Ther. 64: 150-159.
Resnitzky, D., Gossenm, M., Bujard, H. and Reed, S. I. (1994) Acceleration of the G1/S phase transition by expression of cyclins D1 and E with an inducible system. Mol. Cell. Biol. 14: 1669-1679.
Sanwald, P., David, M. and Dow, J. (1996) Use of electrospray ionization liquid chromatography mass spectrometry to study the role of CYP2D6 in the in vitro metabolism of 5-hydroxtryptamine receptor antagonists. J. Chromatography 678: 53-61.
Shimada, T., Yamazaki, H., Mimura, M., Inui, Y. and Guengerich, F. P. (1994) Interindividual variations in human liver cytochrome P-450 enzymes involved in the oxidation of drugs, carcinogens and toxic chemicals: Studies with liver microsomes of 30 Japanese and 30 Caucasians. J. Pharmacol. Exp. Ther. 270: 414-423.
Skoda, R. C., Gonzalez, F. J. and Demierre, A. (1988) Two mutant alleles of the human cytochrome P450 dbl gene (P450 IID1) associated with genetically deficient metabolism of debrisoquine and other drugs. Proc. Natl. Acad. Sci. USA 85: 5240-5243.
Smith, G., Stubbins, M. J., Harries, L. W. and Wolf, C. R. (1998) Molecular genetics of the human cytochrome P450 monooxygenase superfamily. Xenobiotica 28: 1129-1165.
Stricker, B. C. H. (1992) Drug-induced hepatic injury. 2^{a} edición. Elsevier, Amsterdam, Holanda.
Stein, C. A. and Cheng, Y. C. (1993) Antisense oligonucleotides as therapeutic agents. Is the bullet really magical? Science 261:1004-1012
Stuart, G. W., Searle, P. F., Chen, H. Y., Brinster, R. L. and Palmiter, R. D. (1984) A 12-base-pair DNA motif that is repeated several times in metallothionein gene promoters confers metal regulation to a heterologous gene. Proc. Natl. Acad. Sci. USA 81: 7318-7322.
Timbrell, J. A. (1991) Principles of biochemical toxicology. Taylor and Francis, London.
Transon, C., Lecoeur, S., Leemann, T., Beaune, P. and Dayer, P. (1996) Interindividual variability in catalytic activity and immunoreactivity of three major human liver cytochrome P450 isozymes. Eur. J. Clin. Pharmacol. 51: 79-85.
Trapnell, B. C. (1993) Adenoviral vectors for gene transfer. Adv. Drug Del. Rev. 12: 185-199.
Tsutsui, H., Terano, Y., Sakagami, C., Hasegawa, I., Mizoguchi, Y. and Morisawa, S. (1992) Drug specific T cells derived from patients with drug induced allergic hepatitis. J. Immunol. 149:706 716.
Van Doren, K., Hanahan, D. and Gluzman, Y. (1984) Infection of eukaryotic cells by helper-independent recombinant adenoviruses: early region 1 is not obligatory for integration of viral DNA. J. Virol. 50: 606-612.
Venkatakrishnan, K., Greenblatt, D. J., VonMoltke, L. L. and Shader, R. I. (1998) Alprazolam is another substrate for human cytochrome P450-3A isoforms. J. Clin. Psychopharmacol. 18: 256.
Wrighton, S. A., Stevens, J. C., Becker G. W. and VandenBranden, M. (1993) Isolation and characterization of human liver cytochrome P4502C19: correlation between 2C19 and S-mephenytoin 4′-hydroxylation. Arch. Biochem. Biophys. 306 :240-245.
Watkins, P. B. (1990) Role of cytochromes P450 in drug metabolism and hepatotoxicity. Semin. LiverDis. 10: 235-250.
Yang, T. J., Shou, M., Korzekwa, K. R., Gonzalez, F. J., Gelboin, H. V. and Yang, S. K. (1998) Role of cDNA-expressed human cytochromes P450 in the metabolism of diazepam. Biochem. Pharmacol. 55: 889-896.
Zimmerman, H. J. (1982) Hepatotoxicity. Appleton Century Crofts Ed., New York. Zimmerman, H. J. and Maddrey, W. C (1995) Acetaminophen (paracetamol) hepatotoxicity with regular intake of alcohol: analysis of instances of therapeutic misadventure. Hepatology 22:767-773.

1. A method for obtaining a cell model, wherein said model comprises a set of expression vectors that confer to the transformed cells a phenotypic profile of drug biotransformation enzymes comprising:
(a) transforming cells expressing cytochrome reductase with at least one expression vector,
wherein each expression vector comprises a DNA sequence that codes for a different drug biotransformation enzyme, selected from:
(i) a DNA sequence transcribed in the sense MRNA of a drug biotransformation enzyme; and
(ii) a DNA sequence transcribed in the anti-sense MRNA of a drug biotransformation enzyme;
wherein the expression of said DNA sequence in the cells transformed with at least one expression vector confers on the transformed cells a specific phenotypic profile of a drug biotransformation enzyme, and
(b) obtaining cells that transiently express said DNA sequence and present a different phenotypic profile of drug biotransformation enzymes.

2. The method of claim 1, wherein said cells are selected from human or animal cells.

3. The method of claim 2, where in said cells are tumour cells.

4. The method of claim 1, wherein said cells are human cells selected from cells of hepatic, epithelial, endothelial and gastrointestinal type CaCO-2 cells.

5. The method of claim 1, wherein said drug biotransformation enzymes are selected from oxygenases, oxidases, hydrolases and conjugation enzymes.

6. The method of claim 1, wherein said drug biotransformation enzymes are selected from monooxygenases dependent on CYP450, flavin-monooxygenases, sulfo-transferases, cytochrome C reductases, UDP-glucuronyl transferases, epoxide hydrolases and glutathione transferases.

7. The method of claim 1, wherein said DNA sequence coding for a drug biotransformation enzyme comprises at least one DNA sequence from DNA sequences transcribed in the sense MRNA or anti-sense MRNA of CYP450 isoenzymes and DNA sequences transcribed in the sense mRNA or anti-sense mRNA of oxygenases, oxidases, hydrolases and conjugation enzymes involved in drug biotransformation.

8. The method of claim 1, wherein said DNA sequence comprises at least one DNA sequence from DNA sequences transcribed in the sense MRNA or anti-sense MRNA of CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5, GST(A1), and DNA sequences transcribed in the sense MRNA or anti-sense MRNA of flavin-monooxygenases, sulfo-transferases, cytochrome C reductase, UDP-glucuronyl transferase, epoxide hydrolase or glutathione transferase.

9. The method of claim 1, wherein said DNA sequence is a DNA sequence transcribed in the sense mRNA of a Phase I or Phase II drug biotransformation enzyme.

10. The method of claim 1, wherein said DNA sequence is a DNA sequence transcribed in the anti-sense MRNA of a Phase I or Phase II drug biotransformation enzyme.

11. The method of claim 1, wherein said expression vector is selected from viral vectors, liposomes and micellar vehicles.

12. The method of claim 11, wherein said expression vector is chosen from natural and recombinant adenovirus.

13. The method of claim 1, which comprises using variable amounts of at least two said expression vectors comprising DNA sequences coding for the drug biotransformation enzymes selected from Phase I drug biotransformation enzymes and Phase II drug biotransformation enzymes.

14. A method for studying a drug, which comprises placing said drug in contact with a cell model obtained according to the method of claim 1.

15. Use of sense or anti-sense expression vectors of Phase I or Phase II drug biotransformation enzymes in the manipulation of cells expressing cytochrome reductase activity to reproduce the metabolic variability found in humans.

16. A kit comprised of one or more expression vectors coding for the sense and anti-sense MRNA of the Phase I and Phase II drug biotransformation enzymes.

17. A method to confer to a selected cell line the capacity to metabolize xenobiotics in a controllable manner by means of a set of adenoviral expression vectors of Phase I and Phase II drug biotransformation enzymes and cytochrome P450 reductase, comprising the transfection of said cell line with said adenoviral expression vectors to confer to the transfected cells a pre-selected phenotypic profile .

18. The method of claim 17, wherein the selected cell line expresses cytochrome P450 reductase activity, and the set of expression vectors comprises DNA sequences coding for P450 enzymes involved in xenobiotic biotransformation, wherein each expression vector comprises aDNA sequence transcribing for the sense mRNA of a different CYP enzyme.

19. The method of claim 17, wherein the set of expression vectors comprises at least one DNA sequence coding for drug biotransformation enzymes selected from Phase I or Phase II drug biotransformation enzymes, wherein each expression vector comprises a DNA sequence transcribing for the sense MRNA of a different Phase I or Phase II drug biotransformation enzyme.

20. The method of claim 17, wherein the selected cell line contains CYP genes but the cell line does not express CYP reductase and the set of expression vectors comprises DNA sequences coding for at least one of said CYP genes and DNA sequences coding for CYP reductase, wherein each expression vector comprises a DNA sequence transcribing for either the sense MRNA of a CYP enzyme or the sense MRNA of a CYP reductase.

21. A cell model having a phenotypic profile of at least one drug biotransformation enzyme, comprising:
a cell having cytochrome reductase activity, transformed with at least one expression vector comprising a DNA sequence for a drug biotransformation enzyme.

22. The model of claim 21 wherein the DNA sequence is chosen from DNA sequences for oxygenases, oxidases, hydrolases, and conjugation enzymes.

23. The model of claim 21 wherein the DNA sequences are chosen from monooxygenases dependent on CYP450, flavin-monooxygenases, sulfo-transferases, cytochrome C reductases, UDP-glucuronyl transferases, epoxide hydrolases and glutathione transferases.

24. The model of claim 21 wherein the DNA sequences are chosen from CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5, and GST(A1).
